(19) United States
(12) Patent Application Publication
(10) Pub. No.: US 2011/0274670 A1
(43) Pub. Date: Nov. 10, 2011

(54) MESENCHYMAL STEM CELLS WHICH EXPRESS HUMAN HEPATIC GROWTH FACTOR,MANUFACTURING METHOD THEREOF, AND USE THEREOF AS THERAPEUTIC AGENT FOR LIVER DISEASES

(76) Inventors: Myeong Jin Nam, Seoul-city (KR); Sang Koo Lee, Seoul-city (KR)

(21) Appl. No.: 12/997,875

(22) PCT Filed: Mar. 20, 2009
(86) PCT No.: PCT/KR09/01446

§ 371 Date: Dec. 13, 2010

(30) Foreign Application Priority Data
Jun. 13, 2008 (KR) 10-2008-0055690
Dec. 24, 2008 (KR) 10-2008-0132794

(51) Int. Cl. A61K 35/12; C12N 15/85; C12N 5/02; A61P 35/00; C12N 15/867; A61K 31/7088; A61P 1/16; C12N 5/10; C12N 15/861

(52) U.S. Cl. 424/93.21; 435/372; 435/455; 435/404; 514/44 R; 424/93.7

(57) ABSTRACT

The present invention relates to adult stem cells and a manufacturing method thereof. More specifically, the present invention relates to a recombinant expression vector containing a human hepatic growth factor (hHGF) gene, mesenchymal stem cells which are transformed thereby and express the hHGF, a manufacturing method of the mesenchymal stem cells, conditioned media (CM) which is obtained from the transformed cells and proliferates hepatocytes, a culture method of the mesenchymal stem cells producing the same, and the use of the transformed mesenchymal stem cells and their culture media as an agent for preventing and treating liver diseases. The manufacturing method of the mesenchymal stem cells comprises the steps of: isolating and culturing umbilical cord blood-derived mesenchymal stem cells; transforming the mesenchymal stem cells with the recombinant expression vector; and selecting the mesenchymal stem cells. The mesenchymal stem cells, which produce the hHGF in the present invention effectively, proliferate hepatocytes, suppress apoptosis and effectively suppress liver cirrhosis. Therefore, the mesenchymal stem cell can be widely used in preventing and treating various liver diseases.

### TECHNICAL FIELD

The present invention relates to adult stem cells that can be used as an agent for treating a liver disease, and a method for manufacturing the same. More specifically, the present invention relates to a recombinant expression vector containing a human hepatic growth factor (hereinafter it will be called as “hHGF” that is an acronym) gene, mesenchymal stem cells which are transformed thereby and express the hHGF, a manufacturing method of the mesenchymal stem cells, conditioned media (hereinafter it will be called as “CM” that is an acronym) which is obtained from the transformed cells and proliferates hepatocytes, a culture method of the mesenchymal stem cells producing the same, and the use of the transformed mesenchymal stem cells and their culture media as an agent for preventing and treating liver diseases, in which the manufacturing method of the mesenchymal stem cells comprises the steps of: isolating and culturing umbilical cord blood-derived mesenchymal stem cells; transforming the mesenchymal stem cells with the recombinant expression vector; and selecting the mesenchymal stem cells.

### BACKGROUND ART

A liver disease is one of the diseases having a high incidence in Korean, and specifically a cirrhosis of liver and liver cancer that are caused by a chronic hepatitis are one of an important diseases that should surely be overcame. According to Statistical Yearbook of Koreans' Death Cause, the mortality caused by an infectious disease is gradually decreased, while the mortality caused by chronic degenerative diseases including a hepatocirrhosis is increased. In addition, it is reported that the mortality caused by a liver cancer is ranked number one in the world as 23.7 deaths per 100,000 people, and the mortality caused by a chronic liver disease, such as cirrhosis of liver, is ranked number three in the world as 28.8 deaths per 100,000 people. However, there are not satisfactory agents for treating these diseases until now in spite of many researches and effects about chronic liver diseases, such as cirrhosis of liver.

In addition, the chronic liver diseases are generally and partially developed to a liver cancer, in which the route of progress of the chronic liver disease is as follows: the chronic hepatitis is progressed after suffering from a viral or alcoholic hepatitis, and then in many cases, some of chronic hepatitis are progressed to a liver cancer through cirrhosis of liver. When a liver cell is damaged by the cause of autoimmune, hepatitis viruses, and the like, a macrophage performs a phagocytosis in the damaged liver cell; a fibrocyte is moved to the site that is damaged and secrets a collagen, etc to process a fibrosis that is filled with a fiber and regenerating nodule, thereby causing cirrhosis of liver.

A liver function test [AST, ALT, Bilirubin, alkaline phosphatase (ALP) and the line] through a blood test, a serologic marker test for a hepatitis virus, a liver ultrasonography, a peritoneoscopy, a liver biopsy, and the like are used for diagnosing the liver diseases, and an albumin in a blood and a blood coagulation time (Prothrombine time; PT) are measured for estimating the remained liver functions.

Moreover, there has been no cure developed to treat a chronic liver disease. An adjuvant systemic therapy has been only performed through using an immunopotentiator or administrating an antiviral agent in the case of a hepatitis B or a hepatitis C after identifying the cause of the disease. Ara-A, Ara-Amp, acyclovir, suramin, and the like are used as an antiviral agent, and a lamivudine (3TC) is recently used in the clinical trial as a new treatment about a hepatitis B. The lamivudine is an oral antiviral agent as nucleotide derivatives and is used since 1995 in America as AIDS treatment for specifically inhibiting the activity of reverse transcription of acquired immune deficiency virus (HIV). Firstly, Dinstars, et. al. used the lamivudine to the patients with a chronic hepatitis B as a trial. During the administration of the lamivudine, the negativation of HBV DNA is occurred and the level of ALT is normalized in 19% of the patients to continuously maintain the negativation of HBV DNA in a blood. However, it has a disadvantage such that the lamivudine cannot be taken for a long time since it has toxicity. And if stopping the taking of agent, the proliferation of virus that is suppressed is started to back the original state. It was identified that HBV mutant having a resistance to the agent began its existence in the patients, which show the resistance about the administration of lamivudine for a long time, i.e., recent 6 to 12 months.

In addition, an interferon is used as an immunopotentiator for treating a hepatitis B but its effectiveness is very low. However the administration for 3 to 6 months is standard; when administrating for long time, more effective results is expected but it causes a heavy side effect; and when stopping the administration of agent, the proliferation of virus is again increased and then back to the original state. For the treatment of chronic hepatitis B, it is an important that the level of ALT is improved and HBe antigen is negativated. However, even though HBe antigen is negativated in a blood, there are instances that the state of an active hepatitis continues. The adjuvant systemic therapy needs a long period time for treating, as well as its cost is really expensive, but its effectiveness is low and the rate of recurrence is high, and also thyroid disease can be caused by forming an autoantibody or accompanying a side effect.

Specifically, when developing to a liver cancer through a chronic liver disease, a surgical resection or non-surgical treatment, such as a transarterial chemoembolization (TACE), a percutaneous ethanol injection therapy (PEIT), a radio frequency ablasion (RFA), and the like can be used, but an accompanying pain caused by using these therapies and the economical burden are more increased and the effectiveness is no effective. Thus, it requires new approach methods to compensate the problems about the therapies for treating a chronic liver disease and liver cancer until now to develop a remedy in the stage of a chronic liver disease, such as cirrhosis of liver that is a pre-stage of liver cancer. Thus, the therapy for treating all kinds of diseases has been proposed to develop a therapy for using a stem cell.

A mesenchymal stem cell (MSCs) is a stem cell having a multipotent that can be divided to a cell derived from a mosoblast and an adult stem cell can be easily taken from fat, cord blood, peripheral blood and born marrow of adult. Especially, there is no difficulty for using a mesenchymal stem cell isolated from born marrow and cord blood of human as various uses regardless an age. Thus, methods for introducing hepatocyte growth-promoting factors to a cell using a direct or suitable carrier and a gene that expresses hepatocyte growth-promoting factors or active fragment thereof to a mesenchymal stem cell may be used in order to increase the level of hepatocyte growth-promoting factors that are present in a mesenhymal stem cell using a mesenchymal stem cell.

### DISCLOSURE

### [Technical Problem]

The inventors developed a mesenchymal stem cell that are transformed for producing hHGF by manufacturing a recombinant expression vector containing hHGF gene, culturing an isolated mesenchymal stem cell from a cord blood, transforming with each recombinant expression vector and then selecting a cell from the result of continuous effects in order to develop a new agent for treating a liver disease. In addition, the inventors cultured the mesenchymal stem cell for producing CM from the transformed cell and then successfully finished the present invention by identifying the development of the agent for preventing and treating a liver disease through proliferating a hepatocyte, suppressing an apoptosis, and effectively alleviating a condition of cirrhosis using the transformed mesenchymal stem cell and/or CM thereof.

An object of the present invention is to provide a stem cell that can be used as a new agent for treating a liver disease, and a method for producing the same.

In order to achieve the above object, the present invention is to provide mesenchymal stem cells (MSCs) that express hHGF by transforming the mesenchymal stem cells with the recombinant expression vector containing hHGF.

In addition, the present invention is to provide a method for manufacturing the mesenchymal stem cell (MSCs), comprising: (1) isolating a mesenchymal stem cell; (2) sub-culturing the mesenchymal stem cell; (3) transforming the mesenchymal stem cells with the recombinant expression vector containing hHGF gene; and (4) selecting the transformed mesenchymal stem cells that can express hHGF.

In addition, the present invention is to provide a method for culturing the mesenchymal stem cell for manufacturing a conditioned media (CM) having an effect for proliferating a hepatocyte using the mesenchymal stem cells.

Preferably, the mesenchymal stem cell is sub-cultured to 7 to 9 generations, and preferably 8 generations; cultured to reach the cell up to 70 to 90%, and preferably about 80% using the media containing serum; transformed; further cultured for 2 to 10 hours, and preferably 6 hours; and then cultured in the media without a serum for about 3 days.

In addition, the present invention is to provide CM having an effect on proliferating a hepatocyte that is manufactured according to the method for culturing the mesenchymal stem cells.

In addition, the present invention is to provide a pharmaceutical composition having the mesenchymal stem cells (MSCs) as an effective component that can express hHGF for treating a liver disease.

In addition, the present invention is to provide a pharmaceutical composition having CM as an effective component that has an effect for proliferating a hepatocyte for treating a liver disease.

In addition, the present invention is to provide a kit for treating a liver disease, in which the kit is constituted by combining more than one selected from the group consisting of the recombinant expression vector containing hHGF, the mesenchymal stem cell, the transformed mesenchymal stem cell and CM having an effect on proliferating a hepatocyte.

In addition, the present invention is to provide a method for treating a liver disease, in which the method can improve a damaged liver by injecting the conditioned media having an effect on proliferating a hepatocyte or the transformed mesenchymal stem cells in vivo.

### [Technical Solution]

Hereinafter, the present invention will be described in detail as follows.

The present invention is to provide an adult stem cell that expresses a hepatic growth factor (HGF).

The hepatic growth factor (HGF) is also called as a scatter factor (SF) and is a multifunctional heterodimer polypeptide that can be produced by a mesenchymal stem cell. HGF is composed of the constitution containing 34 kDa beta-chain having the similarities to a protease domain of chymotrypsin-like serine protease and 69 kDa alpha-chain containing four kringle domains and N-end finger domain. The human HGF is synthesized to a precursor having 728 amino acids of the type of a single chain that is biologically inactive, and then becomes HGF that is biological active by cutting R494 residue with a specific serum serine protease. The active HGF is a heterodimer that is linked between 69 kDa alpha-chain and 34 kDa beta-chain by disulfide. The preferable nucleotide sequences encoding HGF is as disclosed in a sequence list, in which the base sequence of Sequence No. 1 includes the whole genes, the base sequence of Sequence No. 2 includes only 2.82 kb among the whole genes, and the base sequence of Sequence No. 3 includes only the sequence encoding the HGF gene.

The adult stem cell is an undifferentiated cell that is to differentiate to a cell in a specific organ if it requires. The adult stem cell line may be derived from born marrow, blood, cord blood, liver, skin, gastrointestinal track, placenta or uterus, and it is preferably an adult stem cell derived from cord blood, and more preferably a mesenchymal stem cell derived from cord blood. The adult stem cell derived from cord blood (umbilical cord blood) contains a great quantity of hematopoietic stem cell, and also the stem cell that founds in a born marrow in a born contains various types of adult stem cells, such as a mesenchymal stem cell, a hematopoietic stem cell that can produce a blood and a lymphocyte. Among those cells, the mesenchymal stem cell is easily proliferated in vitro, is a cell that can be differentiated to various types of cells (a fat cell, a cartilage cell, myocyte, and a born cell) and can be used as a target useful for treating a gene or a cell.

The adult stem cell is in an undifferentiated state and has a potential ability on differentiating. However, since the mesenchymal stem cell can be differentiated to various cells derived from a mesoderm, so that it cannot be sure if the injected mesenchymal stem cell is surely differentiated to a parenchymal tissue cell, there is a limit to a method for treating a liver disease using the mesenchymal stem cell. In order to solve the above problem, the inventors were tried to research for developing the method for improving the probability of differentiating to a liver parenchymal cell. Thus, the inventors found out that when introducing the gene encoding HGF to the mesenchymal stem cell, the transformed mesenchymal stem cell has a high probability of differentiating to a liver parenchymal tissue cell as compared with the mesenchymal stem cell and the protein relevant to the liver parenchymal cell is expressed and secreted in a high level.

The present invention is to provide transformed mesenchymal stem cells (MSCs) that can express a human hepatic growth factor (hHGF). Preferably, the mesenchymal stem cell is transformed with the recombinant expression factor containing hHGF and the active fragment thereof, and the recombinant expression factor preferably includes pMEX-HGF, pMSCV-HGF, and the like.

The transformed mesenchymal stem cell was donated to Korean Cell Line Research Foundation in Seoul National University that is an international microorganism depositary authority at Dec. 11, 2009 (Donated No.: KCLRF-BP-00196).

In addition, the present invention is to provide a use for using the adult stem cell expressing HGF as an agent for treating a liver disease.

Specifically, the present invention is to provide a use for using the mesenchymal stem cell (MSCs) expressing hHGF as an agent for preventing and treating a liver disease. The liver disease includes cirrhosis of liver, a liver cancer, hepatitis, and the like.

In addition, the present invention is to provide the recombinant expression vector containing the whole or part active fragment of hHGF gene.

Specifically, the present invention is to provide the recombinant expression vector pMEX-HGF containing the whole gene of hHGF and the transformed E coli transformed by using the same. In addition, the present invention is to provide the recombinant expression vector pMSCV-HGF containing the whole gene of hHGF and the transformed E coli transformed by using the same.

The recombinant expression vector used for the present invention may include all kinds of plasmide vectors, cosmid vectors and virus vectors, and the like in addition to pMEX-neo or pMSCV-neo that are basis of the recombinant expression vector. Among the vectors, the virus vectors are preferably used, and an retrovirus vector, an adenovirus vector, an adeno-associated virus, a herpes simplex virus, and the like including a human immunodeficiency virus (HIV), a murine leukemia virus (MLV), an avian sarcoma leucosis virus (ASLV), a spleen necrosis virus (SNV), a rous sarcoma virus (RSV) and a mouse mammary tumor virus (MMTV) are most preferably used. The range of the present invention may include all the cases such that the whole of hHGF genes or the part active fragments of hHGF genes are injected into the expression vectors to use for transforming the cell including all kinds of stem cells.

In addition, the present invention is to provide a method for manufacturing the mesenchymal stem cell (MSCs), comprising: (1) isolating a mesenchymal stem cell; (2) sub-culturing the mesenchymal stem cell; (3) transforming the mesenchymal stem cells with the recombinant expression vector containing hHGF gene; and (4) selecting the transformed mesenchymal stem cells that can express hHGF.

For the above step (1), it is preferable for isolating the mesenchymal stem cell derived from a human cord blood. Specifically, the human cord blood is isolated, centrifuged, washed with DPBS buffer solution and the like, again centrifuged, and then properly maintained in ACK buffer solution, and the like at a room temperature. Again, the culture media having five times of the buffer solution are added and then centrifuged. Since then, it is treated with LIF buffer solution, and the like, and added with the culture media for stabilizing the cell. The mesenchymal stem cell obtained according to the above ways is preferably used after sub-culturing per 5 days, and after repeating 7 to 8 generations of the above processes, the sub-cultured mesenchymal stem cells are most preferably used.

For the above step (2), the mesenchymal stem cell is preferably used after sub-culturing 7 to 9 generations, preferably 8 generations. For the above step (3), the recombinant expression vector preferably includes pMEX-HGF or pMSCV-HGF that is the recombinant expression vector, and all of the general methods for transforming the cell can be used but it is most preferably transformed by using a nanoparticle as the recombinant expression vector. In addition, for the above step (4), the transformed cell is most preferably selected by using an antibiotic marker including a neomycin.

In addition, in order to introducing the whole or part active fragments of hHGF genes, the above steps can use all of various methods for introducing a gene that are already known in the relevant field, including; a method using a retrovirus, a method using an adenovirus, DNA-calcium precipitation, a method using a liposome, a method using a polyamine-based thing, an electroporation, one of Hanahan method, in which the efficiency is improved by using DMSO (dimethyl sulfoxide) that is an reducing substance in CaCl₂ method, a calcium phosphate precipitation, a protoplast fusion, a stirring using silicon carbide fiber, an agrobacteria-mediated transformation, a transformation using polyethyleneglycol (PEG), dextran sulfate-, lipopectamine- and dry/suppression-mediated transformations, and the like.

Especially, it is most preferable method using an adenovirus. Specifically, the method includes as follows: the recombinant expression vector is manufactured by inserting the whole or part of hHGF genes to the adenovirus expression vector; the transformed packaging cell is selected by transducing the above vector into a packaging virus; the transformed packaging cell is cultured and then again filtered to obtain the adenovirus solution; the mesenchymal stem cell is contaminated by using the adenovirus solution; and then the mesenchymal stem cell that continuously expresses HGF is selected by using the selection marker containing the recombinant adenovirus expression vector.

In addition, the present invention is to provide an agent for preventing and treating a liver disease, containing the mesenchymal stem cell (MSCs) expressing hHGF as an effective component.

The liver diseases objected in the present invention mean all the liver diseases that cause a damage of a liver parenchymal cell, and the liver disease preferably include a chronic and acute A-type, B-type, C-type hepatitis, cirrhosis of liver, hepatocirrhosis, a liver cancer, a fat liver, and the like. Most preferably, it is used for treating cirrhosis of liver derived from hepatitis B virus. The terms “Treatment and prevention” used for the present invention mean all the actions for suppressing or delaying the development of diseases relevant to the liver disease by using the adult stem cell transduced with the gene expressing HGF, and the term, “treatment” means all the actions for beneficially alternating or improving the liver disease by using the cell line.

In addition, the present invention is to provide a culture media of the mesenchymal stem cell that produces the conditioned media (CM) having an effect on proliferating a hepatocyte by using the transformed mesenchymal stem cell.

Specifically, the present invention is to provide a method for culturing the mesenchymal stem cell, comprising: the mesenchymal stem cell is sub-cultured to 7 to 9 generations, and preferably 8 generations; cultured to reach the cell up to 70 to 90%, and preferably about 80% using the media containing serum; transformed; further cultured for 2 to 10 hours, and preferably 6 hours; and then cultured in the media without a serum for about 3 days. The method for culturing can be used for all the stem cells in addition to the mesenchymal stem cell, and the mesenchymal stem cell may include all of the transformed stem cells and untransformed stem cells.

In addition, the present invention is to provide CM having an effect on proliferating a hepatocyte isolated from the transformed mesenchymal stem cell expressing hHGF.

In addition, the present invention is to provide a method for manufacturing CM by manufacturing and culturing the transformed mesenchymal stem cell expressing hHGF in the media without a serum.

In addition, the present invention is to provide the use for using CM of the transformed mesenchymal stem cell expressing hHGF for treating a hepatocyte. The conditioned media (CM) can be used as an agent for treating and preventing various liver diseases including cirrhosis of liver, a liver cancer, a hepatitis, and the like.

According to the present invention, HGF gene is transformed by using the recombinant expression vector that is inserted with hHGF gene to the mesenchymal stem cell isolated from the human cord blood and then hMSCs transformed with HGF is only selectively cultured in order to develop the transgenic adult stem cell for treating cirrhosis of liver. In addition, the effectiveness and stability of the treatment of HGF gene-modified stem cell that is produced through the animal experiment using the animal that is caused with cirrhosis of liver are investigated. Specifically, although various experiment animals are discussed about cirrhosis of liver, but CCl4 or TAA that are reported for relatively surely inducing cirrhosis of liver is injected to the abdominal cavity of the animals to use the experiment animals that are induced with cirrhosis of liver in the present invention. The above experiment animals are divided into a normal group, a group with cirrhosis of liver and a group that is treated with the treatment of HGF gene-modified stem cell for experiments and the tissues and bloods obtained from the experiment animals are performed for a tissue staining, a measurement of total collagen amount, a serum biochemical test, and the like to compare and estimate the effectiveness of the treatment of HGF gene-modified stem cell and the antifibrotic degree.

The present invention is to provide a pharmaceutical composition for treating a liver disease, in which the composition includes the mesenchymal stem cell (MSCs) expressing hHGF as an effective component.

In addition, the present invention is to provide a pharmaceutical composition for treating a liver disease, in which the composition includes the conditioned media having an effect on proliferating a hepatocyte. Preferably, the liver disease includes all of cirrhosis of liver, a liver cancer, a hepatitis, and the like.

The pharmaceutical composition according to the present invention can be used by forming an oral dosage form, such as a powder, a granule, a tablet, a capsule, a suspension, an emulsion, a syrup, an aerosol, and the like, a type of sterile injection solution, an external application, such as an ointment, a suppository, and the like according to the general method that is adjusted for each object for using. The composition may include an excipient and a carrier and the diluents may include lactose, dextrose, sucrose, sorbitol, mannitol, xylitol, erythritol, maltitol, starch, acacias rubber, alginate, gelatin, calcium phosphate, calcium silicate, cellulose, methyl cellulose, non-crystalline cellulose, polyvinyl pyrrolidone, water, methylidroxybenzoate, propylhydroxybenzoate, talc, magnesium stearate, mineral oil, and the like. The solid medicine for administrating orally includes tablet, pill, powder, granule, capsule, and the like and the solid medicine is formed by mixing at least one excipient, for example calcium carbonate, sucrose, lactose, gelatin, and the like in the composition. In addition, a lubricant such as magnesium stearate, talc, and the like are used in addition to the simple excipient. The liquid medicine for administrating orally includes suspension, solution, emulsion, syrup and the like and may include various excipient, for example wetting agent, sweeting agent, aromatic agent, preservatives, and the like in addition to water and liquid paraffin that are simple diluents commonly used. The medicine for a parenteral administration includes a sterile water solution, non-aqueous solution, suspension, the freeze drying form, and suppository. Propylene glycol, polyethylene glycol, vegetable oil such as olive oil, the injectable ester such as ethylolate, and the like may be used as the non-aqueous solution and suspension. The base of parenteral injection may include the conventional additives, such as a solvent, a tonicity adjustment agent, suspension, stabilizer and preservatives. Specifically, it is preferably formed as the type of parenteral injection suitable for injecting to an organ or tissue, and as the type of drying (especially, freeze-drying type) that can be solution capable of injecting by adding with an isotonic sterile solution, a sterile water or a saline. The pharmaceutical composition according to the present invention may be administrated by using various ways, such as a type of oral, intravenous infusion, subcutaneous transfusion, nasal cavity injection, abdominal injection, muscular injection, transdermal administration, and the like. A dosage per one time of the mesenchymal stem cell can be increased according to various factors, such as the disease to be treated, a seriousness of disease, the administration route, a weight of patient, an age of patient, a sec of patient, and the like. Thus, the range of the present invention is not limited to the above dosage.

In addition, the present invention is to provide a kit for treating a liver disease, in which the kit includes the mesenchymal stem cell transformed by a gene of hHGF, or the recombinant expression vector containing hHGF gene and the mesenchymal stem cell. The recombinant expression vector and the mesenchymal stem cell can be individually provided or as the type of the transgenic mesenchymal stem cell transformed by the expression vector. The liver disease preferably includes cirrhosis of liver, a liver cancer, hepatitis, and the like.

In addition, the present invention is to provide a kit for treating a liver disease, in which the kit includes the conditioned media having an effect on proliferating a hepatocyte, in addition to the mesenchymal stem cell and the recombinant expression vector containing hHGF gene, or the transformed mesenchymal stem cell.

In addition, the present invention is to provide a method for treating a liver disease that can prevent all kinds of liver diseases or improve a damaged liver by administrating with the transformed mesenchymal stem cell or the conditioned media having an effect on proliferating a hepatocyte to in vivo. The liver disease preferably includes cirrhosis of liver, a liver cancer, hepatitis, and the like.

### [Advantageous Effects]

As shown in the above description, the present invention a recombinant expression vector containing a human hepatic growth factor (hHGF) gene, mesenchymal stem cells which are transformed thereby and express the hHGF, a manufacturing method of the mesenchymal stem cells, conditioned media which is obtained from the transformed cells and proliferates hepatocytes, a culture method of the mesenchymal stem cells producing the same, and the use of the transformed mesenchymal stem cells for preventing and treating liver diseases, in which the manufacturing method of the mesenchymal stem cells comprises the steps of: isolating and culturing umbilical cord blood-derived mesenchymal stem cells; transforming the mesenchymal stem cells with the recombinant expression vector; and selecting the mesenchymal stem cells. The mesenchymal stem cell producing hHGF according to the present invention can effectively proliferate a hepatocyte and suppress an apoptosis and cirrhosis of liver so that it can be widely used for preventing and treating various liver diseases.

### DESCRIPTION OF DRAWINGS

The above and other objects, features and advantages of the present invention will become apparent from the following description of preferred embodiments given in conjunction with the accompanying drawings, in which:

FIG. 1 is a mimetic diagram showing the restriction enzyme map of the expression vector pMEX-neo used in the present invention;

FIG. 2 is photographs of microscope showing the normal mesenchymal stem cell and the transformed mesenchymal stem cell by using hHGF gene according to the present invention;

FIG. 3 is photographs of microscope showing the normal mesenchymal stem cell and the transformed mesenchymal stem cell by using hHGF gene according to the present invention; A: non-transformed; B: transformed with pGFP3 vector; C: transformed with pGFP3 and pMEX-HGF vector; D: transformed with pGFP3 and pMSCV-HGF vector;

FIG. 4 shows the level of RNA that shows the expression of hHGF in the transformed mesenchymal stem cell by using hHGF gene;

FIG. 5 are the results of Western Blot (a) and ELIZA method (b) in the level of protein that shows the expression of hHGF in the transformed mesenchymal stem cell by using hHGF gene;

FIG. 6 shows the cell viability after treating the cell culture solution containing hHGF that is expressed according to the present invention to the normal hepatocyte (NCTC 1469) and then comparing with a control;

FIG. 7 is a graph showing the results of investigating and comparing with a control about the effect of the cell culture solution containing hHGF protein expressed according to the present invention on an apoptosis;

FIG. 8 are the photographs showing the effect on treating cirrhosis of liver in a liver tissue when administrating the mesenchymal stem cell transformed by hHGF gene according to the present invention to the animal model having cirrhosis of liver;

FIG. 9 to FIG. 11 are photographs of microscope showing the change of liver tissue when injecting the mesenchymal stem cell transformed by using hHGF gene according to the present invention; and

FIG. 12 is a photograph of microscope showing the change of liver tissue when administrating PBS as a control to a liver tissue.

### BEST MODE

Hereinafter, the embodiments of the present invention will be described in detail with reference to Examples.

### Example 1

### Isolation and Culture of Mesenchymal Stem Cell

The mesenchymal stem cells (MSCs) according to the present invention was isolated by using a method combining gradient density centrifugation with plastic adherence using a human umbilical cord blood (HUCB). Specifically, the human umbilical cord blood was centrifuged, the supernatant was removed, the cells in the precipitate were dissolved with DPBS and the cells were again centrifuged. The cells was added with ACK buffer solution and then maintained at a room temperature for 2 minutes. Since then, the cells were added with about 5 times of the above buffer solution and then centrifuged to remove the supernatant. Since then, the cells were added with LIF buffer solution, and then after 1 minute the cells were added with the culture media to filter. The cells obtained from the above procedures were again centrifuged and then cultured as follows:

The mesenchymal stem cell derived from the cord blood isolated from the above procedures was sub-cultured, and then 4^{th} sub-cultured cells (HUCB-derived MSCs) were cultured at 37° C. incubator containing 5% CO₂ by using Dulbecco modified Eagle medium (DMEM) added with 10% fetal bovine serum (FBS), 4 nmol/L L-glutamine, 100 IU/mL penicillin and 100 mg/mL streptomycin. The cells were cultured for 1 to 2 weeks while changing the new medium per 2 days. After the cells were reached to 70% in the culture dish, the cells were isolated from the culture dish by using 0.25% trypsin and 1 mM EDTA, and then sub-cultured in the new culture medium. When the sub-cultured cells were cultured to 7 to 8 generations, the cells were used for the following various experiments. Before using the experiment, the cells was slowly freeze in the culture solution containing 10% DMSO and then stored at −150° C.

### Example 2

### Manufacture of Recombinant Expression Vector Containing HGF Gene and Transformation of Cell

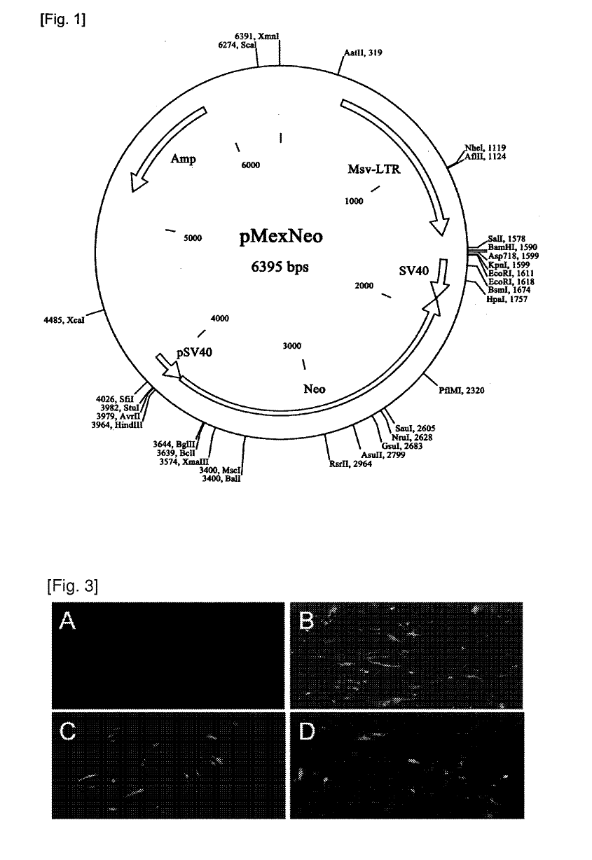

The present invention used HGF (hHGF) gene derived from human as HGF gene in order to manufacture a recombinant expression vector containing HGF gene. The recombinant expression vector pMSCV-HCF and pMEX-HCF according to the present invention were manufactured by inserting the hHGF gene to the conventional pMSCV-neo or pMEX-neo having a tolerance to neomycin (see FIG. 1). At this time, in order to identify the mutation generated when proliferating a gene, the gene sequencing was performed to determine the base sequence by using a primer that is relevant to the suitable part among the gene base sequence. The recombinant expression vectors were transformed to E. coli DH5α to make E. coli Stock and then stored it along with DNA. In addition, in order to transform the cell with the recombinant expression vector, 7 generations sub-cultured cell of the mesenchymal stem cell derived from the human cord blood (HUCB-derived MSCs) was transformed by using the nanoparticle (MNP@SiO2(RITC) PTMA) of the recombinant expression vector pMEX-HGF. The mesenchymal stem cell derived from the cord blood transformed by using hHGF gene (HUCB-derived MSCs) was selected by investigating under the fluorescence microscope whether GFP protein gene that was transferred at the same time when transferring the gene was fluorescent or not (see FIG. 2 and FIG. 3). FIG. 2 is a photograph of microscope showing the normal mesenchymal stem cell and the transformed mesenchymal stem cell by using hHGF gene according to the present invention. FIG. 3 is a photograph of microscope showing the normal mesenchymal stem cell and the transformed mesenchymal stem cell by using hHGF gene according to the present invention. From the above results, it could be identified that the mesenchymal stem cell according to the present invention expressed a fluorescence signal by expressing hHGF and at the same time the shape of cell was changed.

### Example 3

### Investigation of Expressing HGF Gene

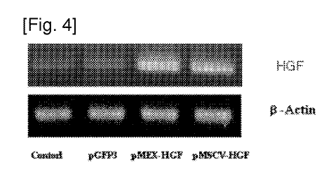

In order to investigate whether HGF gene is expressed in the transformed stem cell or not according to the present invention, the reverse-transcription polymerase chain reaction (RT-PCR) was performed as follows: Firstly, total RNA was obtained by using Trizol (Tel-Test Inc.) from the cell, cDNA was obtained by the reverse transcription reaction using 1^{st} strand cDNA synthesis kit (Roche), and then PCR was performed by using a forward primer of sequence no. 4 (5′-ATGATGATGCTCATGGACCCT-3′) and a reverse primer of sequence no. 5 (5′-CTGGCAAGCTTCATTAAAAC-3′) that are specific to hHGF gene (426 bp). The sample was proliferated in 40 cycles under the conditions of denaturation (95° C., 10 seconds), annealing (57° C., 30 seconds) and synthesis (72° C., 25 seconds). cNDA proliferated from RT-PCT was analyzed and identified on 1.2% agarose gel by using low molecular weight DNA marker (100 bp, Bioneer) (see FIG. 4). FIG. 4 shows the level of RNA that shows the expression of hHGF in the transformed mesenchymal stem cell by using hHGF gene. From the above results, it could be identified that the mesenchymal stem cell could effectively expresses and produce hHGF gene.

### Example 4

### Analysis of Western Blot of hHGF

Western Blot was performed to analysis hHGF expressed according to the present invention as follows: Firstly, the cells were collected from hMSC treated with HGF and hMSC non-treated with HGF by using Trypsin EDTA and then the proteins were obtained from the above cells. The samples were prepared to contain 30 ug of protein, added with the same volume of 2× buffer solution [sample buffer; 0.125M tris (pH 6.8), 6% SDS, 20% glycerol, 0.02% bromophenol blue, 1.44 mM β-mercaptoethanol], boiled at 100° C. for 5 minutes, and then isolated on 10% SDS-polyacrylamide gel. Since then it was transferred to PVDF membrane [polyvinylidene difluoride; Amersham Pharmacia], and added into 5% skim milk dissolved in TBST buffer solution [50 mM Tris (pH 7.5), 150 mM NaCl, 0.1% Tween 20] to react at a room temperature for 1 hour. Since then, it was diluted in a ratio of 1:1000 in 5% skim milk by using a primary antibody, and reacted at a room temperature for 90 minutes, and then washed in 4 times for 15 minutes with TBST buffer solution. Since then, it was diluted in a ratio of 1:2000 in 5% skim milk by using a secondary antibody, and reacted at a room temperature for 60 minutes, and then washed in 4 times for 15 minutes with TBST buffer solution. The specific reaction of the antigen-antibody was investigated and identified by using ECL (Amersham Pharmacia) (See FIG. 5). FIG. 5 are the results of Western Blot (a) and ELIZA method (b) in the level of protein that shows the expression of hHGF in the transformed mesenchymal stem cell by using hHGF gene. From the above result, it could be identified that the mesenchymal stem cell can produce hHGF in a mass amount.

### Example 5

### Preparing the Conditioned Media (CM) Using the Transformed Mesenchymal Stem cell Expressing hHGF

The present invention investigated about the effect of the culture media on suppressing an apoptosis to identify the features of the transformed mesenchymal stem cell expressing hHGF as follows:

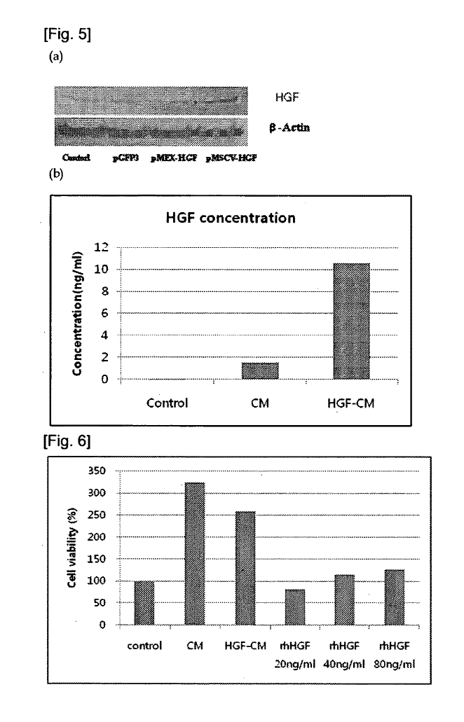

Firstly, the culture solution of the transformed mesenchymal stem cell was collected and then measured the effect of the culture solution on proliferating the normal hepatocyte (NCTC clone 1469) (see FIG. 6). By the way, the culture solution of cell was the conditioned media (CM) without FBS and was specifically produced from the transformed stem cells. Specifically, about 1×10⁴ cells of the mesenchymal stem cell isolated from the cord blood after 8 generations sub-culturing was seeded in MEM media with 10% FBS, and then cultured for 24 hours. Again, the media was changed with the media without serum (serum free MEM) and then the effect on cell viability was investigated by using the media removed from the above change. The detailed conditions for experiment were summarized as the following Table 1 and Table 2. In addition, after finishing the culture time, CM was centrifuged, concentrated by using 50 kD membrane, and then stored at −70° C. before using for the next experiment. From the results, it could be known that CM obtained from the adult stem cell transformed according to the present invention could excellently proliferate a hepatocyte. In addition, as compared with a control, it could be known that hHGF expressed from the adult stem cell according to the present invention had an excellent effect on proliferating a hepatocyte rather than the conventional recombinant rhHGF (see FIG. 6).

In addition, CM produced from the transformed adult stem cell having an effect on suppressing an apoptosis was identified as follows: Firstly, 1×10⁴ cells/ml of the normal hepatocyte (NCTC clone 1469) was seeded and cultured for 24 hours. Again, the media was changed with the media without serum (serum free MEM), treated with 10 ul of GCDC that is a drug for inducing an apoptosis, and then at the same time treated with 10 ul of GCDC and 10 ul of CM, respectively to react for 24 hours. And then MTT analysis was performed to measure the results. From the results, it could be known that the cell viability was excellently increased when adding CM media (see FIG. 7).

TABLE 1
CM culture media transformed with HGF

| | | |
|---|---|---|
| 1 | MSC sub-culture | 8 generations |
| 2 | Culture Media | 10% FBS MEM |
| 3 | CM media | 4 × 10⁵ cells were seeded. After |
| | | reaching about 80%, the cell |
| | | transformation was performed. After |
| | | culturing for 6 hours, the media was |
| | | changed with the media without serum |
| | | and then cultured for 3 days. |
| 4 | Media Amount (Volume) | 6 ml |

TABLE 2
CM culture media without transformation

| | | |
|---|---|---|
| 1 | MSC sub-culture | 8 generations |
| 2 | Culture Media | 10% FBS MEM |
| 3 | CM media | 4 × 10⁵ cells (10% FBS MEM) were |
| | | seeded. After reaching about 70%, |
| | | the media was changed with the media |
| | | without serum and then cultured for 3 |
| | | days. |
| 4 | Media amount (volume) | 6 ml |

### Example 6

### Selection Culture of Mesenchymal Stem Cell Transformed with hHGF Gene

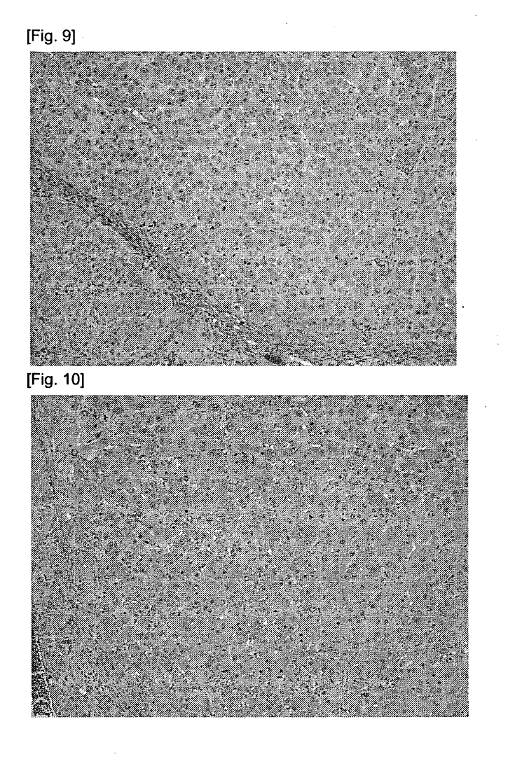

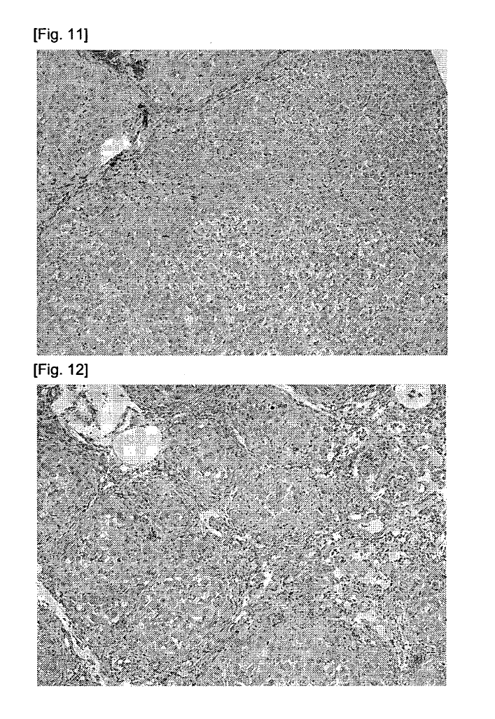

The mesenchymal stem cell expressing hHGF gene (HUCB-derived MSCs) was selectively cultured at 37° C. incubator containing 5% CO₂ by using the DMEM (Dulbecco modified Eagle Medium) media added with 400 ng/mL of neomycin, 10% fetal bovine serum (FBS), 4 nmol/L L-glutamine, 100 IU/mL penicillin, 100 mg/mL streptomycin in order to selectively culture the mesenchymal stem cell transformed with hHGF gene (see FIG. 2). From the results, it could be known that the mesenchymal stem cell according to the present invention can express hHGF and also change at the liver tissue of test animal that was induced with cirrhosis of liver (see FIG. 9 to FIG. 12).

### Example 7

### Investigation of Secreting HGF Protein from Mesenchymal Stem Cell Transformed with hHGF Gene

The amount of HGF protein was measured by ELISA method to investigate the high amount of HGF protein that was secreted from the mesenchymal stem cell transformed with hHGF gene according to the present invention. The adult stem cell derived from the cord blood was used as a control. From the results, it could be known that the mesenchymal stem cell transformed with the recombinant expression vector pMEX-HGF produced 240 ng/ml of the recombinant HGF protein, the recombinant expression vector pMSCV-HGF produced 290 ng/ml of the recombinant HGF protein, and the control produced 35 ng/ml of the recombinant HGF protein.

### Example 8

### Estimation of Effectiveness through Animal Experiment of Mesenchymal Stem Cell Transformed with hHGF Gene

In order to estimate the effectiveness and stability of the mesenchymal stem cell transformed with hHGF gene according to the present invention, the animal experiment was performed as follows: Specifically, after the experiment animal was induced with a liver fibrosis and administrated with the stem cell treatment modified with HGF gene, the change of HGF amount and effect of anti-fibrosis in process of time were estimated through a serum biochemical test, a measurement of total collagen amount and a histopathologic test of liver tissue.

(1) Experiment Animal

Firstly, 12-week aged female wistar rats (230 to 300 g) were divided into three groups of 15 numbers, i.e., PBS treated group (Control), Stem cell treated group and the group that is treated with the stem cell modified with hHGF gene. After the rates in all the groups were induced with cirrhosis of liver, the rates were administrated with PBS, Stem cell, hHGF gene-modified stem cell while the rats were freely taken with food and water giving a night and day.

(2) Inducement of a Liver Fibrosis (Cirrhosis)

CCl₄ and/or TTA were diluted and injected into the abdominal of animals to induce a liver fibrosis (1 ml/kg, three times/week administration). All the animal groups were induced with cirrhosis of liver, and earlier, the animals randomly selected from each group were sacrificed to collect organs and serum. In addition, histological and hematological tests were performed using the samples obtained from the above to identify the successful induction of cirrhosis of liver.

(3) Collection and Store of Blood Sample

The animals were etherized, the blood taken from the caval vein was maintained at 4° C. for at least 4 hours and then centrifuged to investigate the liver tissue of the animals. The serum obtained from the above was stored at −20 in a freezer to use for a hematological and biochemical test.

(4) Collection of Liver Tissue and Observation by Using an Optical Microscope

In order to observe the liver tissue of the above animals, the center of the external right lobe and posterior lobe of liver was cut and fixed in 10% neutral formalin solution. And the tissue specimen having 5 um size was obtained by embedding in paraffin through the dehydration of alcohol. The tissue specimen obtained from the above was stained with a hematoxylin and eosin, and Masson's Trichrome to perform the histopathologic test for estimating the level of liver fibrosis (cirrhosis) and the level of inflammation of tissue.

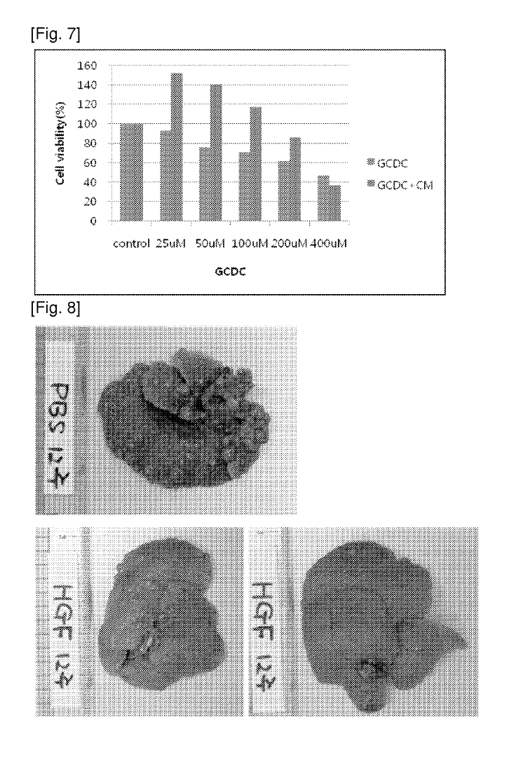

FIG. 8 are the photographs showing the effect on treating cirrhosis of liver in a liver tissue when administrating the mesenchymal stem cell transformed by hHGF gene according to the present invention to the animal model having cirrhosis of liver. FIG. 9 to FIG. 11 are photographs of microscope showing the change of liver tissue when injecting the mesenchymal stem cell transformed by using hHGF gene according to the present invention. FIG. 12 is a photograph of microscope showing the change of liver tissue when administrating PBS as a control to a liver tissue. From the above results, it could be known that the mesenchymal stem cell transformed with hHGF gene according to the present invention has an excellent effect for treating cirrhosis of liver when administrating the animal model having cirrhosis of liver.

(5) Measurement of Collagen Amount in a Tissue

In order to measure total collagen amount in a tissue of the above experiment animals, the liver tissue was hydrolyzed with hydrochloric acid and then taken in a certain amount. And then the hydrochloric acid was evaporated and then the residue was dissolved in isopropyl alcohol to oxidize with chloramines-T. At this time, Ehrlich's reagent solution (p-dimethylaminobenzaldehyde) was used as a color former and measured at the absorbance of 558 nm to calculate total collagen amount in a tissue. From the obtained results, it could be shown that total collagen amount in a tissue was increased.

(6) Serum Biochemical Test

In order to perform the serum biochemical test of the above experiment animals, the collected blood was maintained at a room temperature for 30 minutes in a 10 ml tube to coagulate, and then centrifuged (3,000 rpm for 30 minutes). The amounts of alanine transaminase (alanine; ALT), aspartate transaminase (AST), alkaline phosphatase (ALP) and total bilirubin (TB) were measured in the serum obtained from the above procedures by using RA-XT (Technicon, Ltd., U.S.A.).

TAA drug was administrated for 8 weeks and then the serious damage of liver was identified through the measurements of GOT and GPT. The rats were divided into three groups. The first group was administrated only PBS that is a solvent as a control, the second group was injected with the stem cell containing HGF gene, and the third group was administrated only with the stem cell. PBS and the stem cells were administrated and then TAA was administrated for 4 weeks. And then the damage of liver was induced and then sacrificed. The rats in the group that was only administrated with PBS shown that the livers were continuously damaged and then progressed to a liver cancer. However, the rats in the group that was injected with the stem cell expressing hHGF gene shown that the progression of disease was slow or it was tend to recover, so that there was 50 to 60% treatment effect (see FIG. 8).

(7) Statistical Analysis

In order to statistically analysis with the above results, Student's t-test was performed, and a median and standard deviation were indicated. P-value was obtained to verify significance (p<0.05).

1. A mesenchymal stem cell expressing a human hepatic growth factor (hHGF) transformed with a recombinant expression vector containing hHGF gene.

2. The mesenchymal stem cell (donated No. KCLRF-BP-00196) of claim 1, wherein the recombinant expression vector containing the hHGF gene is pMEX-HGF.

3. The mesenchymal stem cell of claim 1, wherein the recombinant expression vector containing the hHGF gene is pMSCV-HGF.

4. The mesenchymal stem cell of claim 1, using for preventing and treating all kinds of liver diseases including cirrhosis of liver.

5. The method for manufacturing the mesenchymal stem cell according to claim 1, comprising:
(1) isolating a mesenchymal stem cell;
(2) sub-culturing the mesenchymal stem cell;
(3) transforming with the recombinant expression vector containing hHGF gene; and
(4) selecting the transformed mesenchymal stem cell expressing hHGF.

6. The method of claim 5, wherein in the step (1), the mesenchymal stem cell is derived from an umbilical cord blood.

7. The method of claim 5, wherein in the step (2), the mesenchymal stem cell is sub-cultured to 7 to 9 generations, and preferably 8 generations.

8. The method of claim 5, wherein in the step (3), the recombinant expression vector includes the recombinant retrovirus expression vector or recombinant adenovirus expression vector, and preferably the recombinant expression vector pMEX-HGF or the recombinant expression vector pMSCV-HGF.

9. The method of claim 5, wherein in the step (3), the recombinant expression vector is transformed with a nanoparticle.

10. A method for culturing a mesenchymal stem cell producing a conditioned media (CM) having an effect on proliferating a hepatocyte using the mesenchymal stem cell of claim 1.

11. The method for culturing of claim 10, wherein the mesenchymal stem cell is sub-cultured to 7 to 9 generations, and preferably 8 generations; cultured to 70 to 90%, and preferably about 80% using the media with a serum; transformed; again cultured for 2 to 10 hours, and preferably for 6 hours; and then cultured in the media without a serum for 3 days.

12. A conditioned media (CM) having an effect on proliferating a hepatocyte, produced according to the method for culturing the mesenchymal stem cell of claim 10.

13. The conditioned media (CM) of claim 12, using for preventing and treating various liver diseases containing cirrhosis of liver.

14. A pharmaceutical composition for treating a liver disease, wherein the composition includes the mesenchymal stem cell (MSCs) expressing hHGF according to claim 1 as an effective component.

15. The pharmaceutical composition for treating a liver disease according to claim 14, wherein the liver disease includes cirrhosis of liver, a liver cancer and hepatitis.

16. A pharmaceutical composition for treating a liver disease, wherein the composition includes the conditioned media having an effect on proliferating a hepatocyte of claim 12.

17. A kit for treating a liver disease, wherein the kit is composed of the combination of more than one selected from the group consisting of the recombinant expression vector containing hHGF gene, the mesenchymal stem cell, the transformed mesenchymal stem cell and the conditioned media (CM) having an effect on proliferating a hepatocyte.

18. A method for improving a liver disease, improving the damaged liver by administrating a transformed mesenchymal stem cell and the conditioned media having an effect on proliferating a hepatocyte of claim 12 to in vivo.


SEQUENCE LISTING (5 sequences)

SEQ ID NO: 1 - DNA, 5896 bp, Homo sapiens
cacacaacaaacttagctcatcgcaataaaaagcagctcagagccgactggctcttttaggcactgactccgaacaggattctttcacccaggcatctcctccagagggatccgccagcccgtccagcagcaccatgtgggtgaccaaactcctgccagccctgctgctgcagcatgtcctcctgcatctcctcctgctccccatcgccatcccctatgcagagggacataagaaaagaagaaatacaattcacgaattcaaaaaatcagcaaagactaccctaatcaaaatagatccagcactgaagataaaaaccaaaaaagtgaatactgcagaccaatgtgctaatagatgtactaggaataatggacttccattcacttgcaaggcctttgtttttgataaagcgagaaaacaatgcctctggttccccttcaatagcatgtcaagtggagtgaagaaagaatttggccatgaatttgacctctatgaaaacaaagactacattagaaactgcatcatcggtaaaggacgcagctacaagggaacagtatctatcactaagagtggcatcaaatgtcagccctggagttccatgataccacacgaacacagctttttgccttcgagctatcggggtaaagacctacaggaaaactactgtcgaaatcctcgaggggaagaagggggaccctggtgtttcacaagcaatccagaggtacgctacgaagtctgtgacattcctcagtgttcagaagttgaatgcatgacctgcaatggggagagttatcgaggtctcatggatcatacagaatcaggcaagatttgtcagcgctgggatcatcagacaccacaccggcacaaattcttgcctgaaagatatcccgacaagggctttgatgataattattgccgcaatcccgatggccagccgaggccatggtgctatactcttgaccctcacacccgctgggagtactgtgcaattaaaacatgcgctgacaatactgtaaatgatactgatgttcctatggaaacaactgaatgcatccaaggtcaaggagaaggctacaggggcactgccaataccatttggaatggaattccatgtcagcgttgggattctcagtatcctcacaagcatgacatgactcctgaaaatttcaagtgcaaggacctacgagaaaattactgccgaaatccagatgggtctgaatcaccctggtgttttaccactgatccaaacatccgagttggttactgctcccaaattccaaactgtgatatgtcaaatggacaagattgttatcgtgggaatggcaaaaattatatgggcaacttatcccaaacaagatctggactaacgtgttcaatgtggaacaagaacatggaagacttacaccgtcatatcttctgggaaccagatgcaagtaagctgaatgagaattactgccgaaatccagatgatgatgctcatggaccctggtgctacacgggaaatccactcattccttgggattattgccctatttctcgttgtgaaggtgataccacacctacaatagtcaatttagaccatcctgtaatatcttgcgccaaaacgaaacaactgcgagttgtaaatgggattccaacacgaacaaatgtaggatggatgattagtttgagatacagaaataaacatatctgcggaggatcattgataaaggaaagttgggttcttactgcacgacagtgtttcccttctcgagacttgaaagattatgaggcttggcttggaattcatgatgtccatggaagaggagaggagaaacgcaaacaggttctcaatgtttcccagctggtatatggccctgaaggatcagatctggttttaatgaagcttgccagacctgctgtcctggatgattttgttaatacaattgatttacctaattatggatgcacaattcctgaaaagaccagttgcagtgtttatggctggggctacactggattgatcaactatgatggtctattacgagtggcacatctctataaatgggaaatgagaaatgcagccagcatcaccgagggaaggtgactctgaatgagtctgaatatgtgctggggctgagaagattggatcaggaccatgtgagggggattatggtggcccacttgtttgtgagcaacataaaatgagaatggttcttggtgtcattgttcccggccgtggatgcgccattccaaatcgtcctggtatttttgtccgagtagcatattatgcaaaatggatacacaaaattattttaacatataaggtaccacagtcatagctgaagtaagtgtgtctgaagcacccaccaatacaactgtcttttacatgaagatttcagagaatgtggaattaaaaataccacttacaacaatcctaagacaactactggagagtcatgtttgttaaaattctcattaatgtttatgggtgttttctgttgttttgtttgtcagtgttattttgtcaatgttgaagtgaattaaggtacatgcaagtgtagtaacatatctcctgaagatacttgaatggattaaaaaaacacacaggtataattgctggataaagattttgtggggaaaaaatcaattaatctctctaagctgctttctgaggttggtttcttaataatgagtaaaccataaattaaatgttattttaacctcaccaaaacaatttataccttgtgtccttaaattgtaccctatattaaattatattacatttcatatgctatatgttatagttcattcatttctcttcaccatgtatcctgcaatactggtacacgaacacactttttacaaaaccacatacccatgtacacatgcctaggtacacatgtacatgcactacagtttaaattatgatgtacttaatgtaacctctaaatattttagaagtatgtacctatagttttacctcaaaaaaatagaaatctctaaagaccagtagaaatattaaaaaatgatgcaaaatcaaaatgagtggctaattctccatacgtaatctgcagatgatcttctctggttgacattttacgtgtggccatcaccccgggttaaataacacctaatctaggtgtttacatgtattcaatatcctagtttgtttcatgtagtttctaattcttaaaggaaagagggtaataattctatttgtgtaatttgtttcctccaaacttaaggccacttatttacacaagatatttgtatgtctactttcctaaagcatttcttcagtgctcagatcagtgtctaattgaagaagattaaaactgctttggtcattaaaaacgtatttaaataggttaattctaagacttgctgctgtgattgacttctagctcactgcctttaaattttaaaaaatttaagaggaaaattttcatgtctccaaagttttataaatacccttcatcaagtcatgcattaaagtatatattagagaaaaaaaaatacttttctcaacctggaagattttagcctaataaagtttttttgaagtaaaagaaaacttgtaaagggaaagaaactagtttgtctaaactctgtattcattttttttttttttgaagtacagtggaatctgttgaatcagatattttatcaagatatctttattttttcttatttcatttttacaaagatcactcccaatgccatatgtaatagacatttaaatttcgtgttctgtatgacagccaaatgatcatatttatcattgtatttgtcatgtttagctaaaaatcatgtattgttgagaaatagaataacaaaaagtaataggataggctttgaatttttgcaaaaaatcttcctgtacaaaacatctttaaaaataattttttgagtggtgtgaatctagtattcccatttctctgatttagttttcttgagtgatttttatcaaggctaagtccccaaatgattccctaacagctctttagaataccgtttaatctggactaaaatggttttaagtttatggagagtttagtccacagaactaactggacttctggcggcaagtccagaaatgcttatacaaatttttttttcataataagatatgtgctggtatcaaggaacttaaagtggaagcaaaaagacatccaagtagttgctagtctccatcatcttatctgattgtatttctcttttccttatataatacaccattttcataagaacacctagaaatttcaagagtatattgccaaaatataaagtatatttcctagtttcttctggctgaaccagtgaaattttattgttgcatattaatgatatttttaaaacttttataaaaattgtcatacttttaaatactcacattttaaaaatacttcttttatgactcttcctctaaatttcctggaaatacagataaagattagctagatacaagatgcagctaagtatttagacattttgagcccagtatttttcattttattaaaggctaaaaacaataccaccaataaatcatcaaacaaactgtacaaaataattctgtctttgggaggctccttttgtgatagagggacatgggtggaattgacaatgaaagttagatgaacaaggtccgtgttattttaggtagtagaacagggtagagtcatgtcattatttgcgggcggaagatactatttaccacgtgttctttgctgaatcaattattaaacatttttaaaaatccaattatccactttattttgtgtcattgacaaaaggatcttttaagtcagaggtttcaatgtgatttttggcttggctgtttgaataatggttatgtactgttataattgtagacattttctcatgtctaccaggaattgaagtgtaaaactaaaatatttttcataatgcctctgccgtgcggaaggaatgataatccttttgtatacttctttaattttattgtaaaatgtgtaatgacttttacctatatgctgtgggcaggtcctcagtaaaatctattgagtcaatttctagtattaataggcttttgcttgctatctaagtgtttcaaattatgggaagtgtgagacactggaaggcaagaaaattaacaataatggcatgtgatagcaaaattgtatttcacttattcctgtgaatatttcttgttggtaccaatggtactgtacaaagtgaatgttatagccacaacattctcttgaaaagaacactgtcaagaagtgggaaattgctgtcaggcatttcgttgttgtttttaaactttttaaaaaagaaatactggttttgcaagatagagatcatgaggtaaataattttaataagctcttatactaaaaagccttaaatcgatttactgagattcaaaacatactattataatcaattatatcccatatatgtaggcaaactcatttaaaaaataaaattaattttggtaaaagtacatagtgtttgtttttaaaatacataattttaaaataaatcgcttgtcatgataaagtccaaaaagaagttatctttcaatattcaactaagtttggagctaagaatttactaatacaaaaaaaagttaaaatgttttggaccatatatatcttgacagtgtaacttttaagtaggctcatttccatttgcacagaaagtttctgtctttaggaaactgaaaatgaaatactgtggatgttatgactgtttgtcttctatgtaaataggaaattaataagctgcctattgagtggtatagctgtatgcttacccaaaaaagggaacactgtggttatgacttgtattataaactttctgtagttaataaagttgttatttttataaccatgattatatattattattaataaaatattttatcgaaatgct

SEQ ID NO: 2 - DNA, 2820 bp, Homo sapiens
gggagttcagacctagatctttccagttaatcacacaacaaacttagctcatcgcaataaaaagcagctcagagccgactggctcttttaggcactgactccgaacaggattctttcacccaggcatctcctccagagggatccgccagcccgtccagcagcaccatgtgggtgaccaaactcctgccagccctgctgctgcagcatgtcctcctgcatctcctcctgctccccatcgccatcccctatgcagagggacaaaggaaaagaagaaatacaattcatgaattcaaaaaatcagcaaagactaccctaatcaaaatagatccagcactgaagataaaaaccaaaaaagtgaatactgcagaccaatgtgctaatagatgtactaggaataaaggacttccattcacttgcaaggcttttgtttttgataaagcaagaaaacaatgcctctggttccccttcaatagcatgtcaagtggagtgaaaaaagaatttggccatgaatttgacctctatgaaaacaaagactacattagaaactgcatcattggtaaaggacgcagctacaagggaacagtatctatcactaagagtggcatcaaatgtcagccctggagttccatgataccacacgaacacagctttttgccttcgagctatcggggtaaagacctacaggaaaactactgtcgaaatcctcgaggggaagaagggggaccctggtgtttcacaagcaatccagaggtacgctacgaagtctgtgacattcctcagtgttcagaagttgaatgcatgacctgcaatggggagagttatcgaggtctcatggatcatacagaatcaggcaagatttgtcagcgctgggatcatcagacaccacaccggcacaaattcttgcctgaaagatatcccgacaagggctttgatgataattattgccgcaatcccgatggccagccgaggccatggtgctatactcttgaccctcacacccgctgggagtactgtgcaattaaaacatgcgctgacaatactatgaatgacactgatgttcctttggaaacaactgaatgcatccaaggtcaaggagaaggctacaggggcactgtcaataccatttggaatggaattccatgtcagcgttgggattctcagtatcctcacgagcatgacatgactcctgaaaatttcaagtgcaaggacctacgagaaaattactgccgaaatccagatgggtctgaatcaccctggtgttttaccactgatccaaacatccgagttggctactgctcccaaattccaaactgtgatatgtcacatggacaagattgttatcgtgggaatggcaaaaattatatgggcaacttatcccaaacaagatctggactaacatgttcaatgtgggacaagaacatggaagacttacatcgtcatatcttctgggaaccagatgcaagtaagctgaatgagaattactgccgaaatccagatgatgatgctcatggaccctggtgctacacgggaaatccactcattccttgggattattgccctatttctcgttgtgaaggtgataccacacctacaatagtcaatttagaccatcccgtaatatcttgtgccaaaacgaaacaattgcgagttgtaaatgggattccaacacgaacaaacataggatggatggttagtttgagatacagaaataaacatatctgcggaggatcattgataaaggagagttgggttcttactgcacgacagtgtttcccttctcgagacttgaaagattatgaagcttggcttggaattcatgatgtccacggaagaggagatgagaaatgcaaacaggttctcaatgtttcccagctggtatatggccctgaaggatcagatctggttttaatgaagcttgccaggcctgctgtcctggatgattttgttagtacgattgatttacctaattatggatgcacaattcctgaaaagaccagttgcagtgtttatggctggggctacactggattgatcaactatgatggcctattacgagtggcacatctctatataatgggaaatgagaaatgcagccagcatcatcgagggaaggtgactctgaatgagtctgaaatatgtgctggggctgaaaagattggatcaggaccatgtgagggggattatggtggcccacttgtttgtgagcaacataaaatgagaatggttcttggtgtcattgttcctggtcgtggatgtgccattccaaatcgtcctggtatttttgtccgagtagcatattatgcaaaatggatacacaaaattattttaacatataaggtaccacagtcatagctgaagtaagtgtgtctgaagcacccaccaatacaactgtcttttacatgaagatttcagagaatgtggaatttaaaatgtcacttacaacaatcctaagacaactactggagagtcatgtttgttgaaattctcattaatgtttatgggtgttttctgttgttttgtttgtcagtgttattttgtcaatgttgaagtgaattaaggtacatgcaagtgtaataacatatctcctgaagatacttgaatggattaaaaaaacacacaggtatatttgctggatgataaagatttcatgggaaaaaaaatcaattaatctgtctaagctgctttctgatgttggtttcttaataatgagtaaaccacaaattaaatgttattttaacctcaccaaaacaatttataccttgtgtccctaaattgtagccctatattaaattatattacatttcaaaaaaaaaaaaaaaa

SEQ ID NO: 3 - DNA, 2187 bp, Homo sapiens
atgtgggtgaccaaactcctgccagccctgctgctgcagcatgtcctcctgcatctcctcctgctccccatcgccatcccctatgcagagggacataagaaaagaagaaatacaattcacgaattcaaaaaatcagcaaagactaccctaatcaaaatagatccagcactgaagataaaaaccaaaaaagtgaatactgcagaccaatgtgctaatagatgtactaggaataatggacttccattcacttgcaaggcctttgtttttgataaagcgagaaaacaatgcctctggttccccttcaatagcatgtcaagtggagtgaagaaagaatttggccatgaatttgacctctatgaaaacaaagactacattagaaactgcatcatcggtaaaggacgcagctacaagggaacagtatctatcactaagagtggcatcaaatgtcagccctggagttccatgataccacacgaacacagctttttgccttcgagctatcggggtaaagacctacaggaaaactactgtcgaaatcctcgaggggaagaagggggaccctggtgtttcacaagcaatccagaggtacgctacgaagtctgtgacattcctcagtgttcagaagttgaatgcatgacctgcaatggggagagttatcgaggtctcatggatcatacagaatcaggcaagatttgtcagcgctgggatcatcagacaccacaccggcacaaattcttgcctgaaagatatcccgacaagggctttgatgataattattgccgcaatcccgatggccagccgaggccatggtgctatactcttgaccctcacacccgctgggagtactgtgcaattaaaacatgcgctgacaatactgtaaatgatactgatgttcctatggaaacaactgaatgcatccaaggtcaaggagaaggctacaggggcactgccaataccatttggaatggaattccatgtcagcgttgggattctcagtatcctcacaagcatgacatgactcctgaaaatttcaagtgcaaggacctacgagaaaattactgccgaaatccagatgggtctgaatcaccctggtgttttaccactgatccaaacatccgagttggttactgctcccaaattccaaactgtgatatgtcaaatggacaagattgttatcgtgggaatggcaaaaattatatgggcaacttatcccaaacaagatctggactaacgtgttcaatgtggaacaagaacatggaagacttacaccgtcatatcttctgggaaccagatgcaagtaagctgaatgagaattactgccgaaatccagatgatgatgctcatggaccctggtgctacacgggaaatccactcattccttgggattattgccctatttctcgttgtgaaggtgataccacacctacaatagtcaatttagaccatcctgtaatatcttgcgccaaaacgaaacaactgcgagttgtaaatgggattccaacacgaacaaatgtaggatggatgattagtttgagatacagaaataaacatatctgcggaggatcattgataaaggaaagttgggttcttactgcacgacagtgtttcccttctcgagacttgaaagattatgaggcttggcttggaattcatgatgtccatggaagaggagaggagaaacgcaaacaggttctcaatgtttcccagctggtatatggccctgaaggatcagatctggttttaatgaagcttgccagacctgctgtcctggatgattttgttaatacaattgatttacctaattatggatgcacaattcctgaaaagaccagttgcagtgtttatggctggggctacactggattgatcaactatgatggtctattacgagtggcacatctctatataatgggaaatgagaaatgcagccagcatcaccgagggaaggtgactctgaatgagtctgaaatatgtgctggggctgagaagattggatcaggaccatgtgagggggattatggtggcccacttgtttgtgagcaacataaaatgagaatggttcttggtgtcattgttcccggccgtggatgcgccattccaaatcgtcctggtatttttgtccgagtagcatattatgcaaaatggatacacaaaattattttaacatataaggtaccacagtcatag

SEQ ID NO: 4 - DNA, 21 bp, Artificial Sequence
  Forward primer sequence used in RT-PCR
atgatgatgctcatggaccct

SEQ ID NO: 5 - DNA, 21 bp, Artificial Sequence
  Reverse primer sequence used in RT-PCR
ctggcaagcttcattaaaacc